(19) United States
(12) Patent Application Publication
(10) Pub. No.: US 2004/0253716 A1
(43) Pub. Date: Dec. 16, 2004

(54) Bioreactor for cultivating cells on a matrix

(76) Inventors: Volker Jaeger, Braunschweig (DE); Marc Barthold, Hannover (DE)

Correspondence Address: PENDORF & CUTLIFF, 5111 Memorial Highway, Tampa, FL, 33634-7356, US

(21) Appl. No.: 10/816,113

(22) Filed: Apr. 1, 2004

(30) Foreign Application Priority Data
May 16, 2003 (WO) PCT/DE03/01255

(51) Int. Cl. C12M 1/02

(52) U.S. Cl. 435/299.2; 435/295.3; 435/302.1

(57) ABSTRACT

A bio-reactor for cultivating cells, preferably mammalian cells, within a porous support matrix material, the bio-reactor having an inner vessel disposed within an outer vessel, and a matrix carrier containing a support matrix disposed within the inner vessel. The support matrix is provided with a supply of cultivation medium through an inlet and an outlet boring of the matrix carrier.

### FIELD OF THE INVENTION

The present application relates to bioreactor systems useful for cultivating cells, especially mammalian cells within a matrix material. In the invention, the matrix material is used as a scaffold, within which mammalian cells can grow and to which they can adhere.

### BACKGROUND TO THE INVENTION

In order to allow an adequate supply of nutrients, oxygen and further ingredients of a growth medium to the cells, as well as an inflow and ingrowth of cells into the matrix, the matrix to be used in connection with this invention offers interconnected interstices and pores.

A bioreactor for cultivating cells is known from German patent application DE 197 10 65 A1, describing a fermenter useful for maintaining cells in suspended culture. The bioreactor comprises an outer vessel, forming a thermostatic environment for the inner vessel, which is shaped as a frustrum of a cone with its smaller diameter forming the closed bottom portion and its larger diameter forming the open top portion, sealable by a lid. The frustrum shape allows the cells to be cultivated in a small volume of medium in the bottom portion of the inner vessel while the large diameter of the top portion allows the introduction of a sufficiently large number of sampling and supply pipes as well as probes which are fixed within the lid sealing the top portion.

From U.S. Pat. No. 5,190,878 an apparatus for the cultivation of cells is known, comprising a chamber having an inlet port and an outlet port for medium and a flat support arranged within the flow of medium. The flat support is to serve as a substrate for the growth of mammalian cells.

Goldstein et al. disclose in Biomaterials 22 (2001), pages 1279-1288, a flow through bioreactor chamber for cultivating osteoblastic cells. The chamber includes a scaffold arranged such that it is perfused by cell growth supporting medium in order to allow the ingrowth of the cells into the scaffold. The medium perfusing the scaffold enters one side of the chamber holding the scaffold and leaves the chamber on the opposite side of the chamber after being forced to perfuse the scaffold.

When cultivating mammalian cells, especially osteoblastic cells, the costs of the cell growth supporting medium are an important contributor to the overall production costs. In view of the above known state of art it is the object of the invention to provide a bioreactor system which allows to efficiently cultivate mammalian cells to grow on and within a support matrix or scaffold while utilizing a reduced volume of cultivation medium.

It is a further object of the invention to provide a bioreactor system useful for cultivating mammalian cells, especially osteoblastic cells on a support matrix or scaffold having a pre-adapted three-dimensional shape corresponding to a defect in bone. It is desired to provide a bioreactor system capable of cultivating human or humanized osteoblastic cells throughout the body of a support matrix which has been arbitrarily shaped according to the defect of a bone. The resulting support matrix which has been populated by osteoblastic cells is useful for filling bone defects in order to promote healing or bridging gaps of bone, occuring for example after tumor removal.

### SUMMARY OF THE INVENTION

The above objects are achieved by the bioreactor of the invention comprising an inner vessel in the shape of a frustrum of a cone with its smaller diameter portion forming the closed bottom end of the inner vessel and the larger diameter portion being coverable by a lid arrangement to receive the penetrating probes, e.g. pH probes, temperature probes, level probes, probes for dissolved oxygen and pipes into the inner vessel in a sealed manner. The inner vessel is temperature controlled, for example by a thermostatic bath contained in an outer vessel embracing the outer surface of the inner vessel. A matrix carrier is provided for receiving the support matrix therein, which matrix carrier is provided with an inlet boring at a first end and an outlet boring at a second end opposite the first end. The inlet boring leads to the interior space of the matrix carrier, the outlet boring is connected to the first end of a carrier pipe, fixedly arranged within the lid arrangement. The carrier pipe provides a duct for medium to flow from the matrix carrier through the lid arrangement to the second end of the carrier pipe, positioned outside of the inner vessel. The medium is to be returned to the inner vessel via an inlet port penetrating the lid arrangement. The line between the exit port of the carrier pipe and the inlet port is provided by a circulating duct, for example connecting tubing and/or pipeline.

The flow of the cultivation medium into the inlet boring of the matrix carrier, through the interior volume of the matrix carrier, out of its outlet boring, then via the carrier pipe and circulating duct to the inlet port back into the inner vessel is promoted by a pump, arranged at or within the circulating duct.

According to the invention, the interior volume of the matrix carrier between inlet and outlet borings is shaped such that the contoures of its interior correspond to the outer contours of the support matrix, preferably with additional inlet and outlet regions having an essentially conical shape to allow for an even distribution of medium across the adjacent surface of the support matrix. The contoured fit of the matrix carrier around a support matrix to be contained therein allows for a forced flow of medium through the entire cross-section of the support matrix, perfusing its complete volume.

Preferably, the matrix carrier is formed such that its interior surface is adjacent the support matrix in positive fit on all its surface regions except for the inlet and outlet regions in fluid contact with the inlet and outlet borings, respectively. In order to ensure a perfusion of the support matrix and avoid medium to flow inside the matrix carrier around the support matrix, it is preferred that the interior surface of the matrix carrier is in contact with the surface of the support matrix on at least a closed circumferential line of contact around the support matrix, the line of contact being positioned between the inlet and outlet borings of the matrix carrier.

The line of contact between the matrix carrier and the support matrix may also be provided by an additional elastic sealing arranged between the latter, for example an O-ring.

The present invention provides a bioreactor system capable of fixedly holding a support matrix within a matrix carrier which is designed such that the body of the support matrix is perfused to an extent to allow the growth of mammalian cells within the cavities and pores of the matrix material, essentially avoiding the limitation of the cells from the supply with nutrients and oxygen.

Furthermore, the bioreactor system according to the present invention effectively utilizes only a small volume of medium necessary for cell cultivation, thus making cultivation of mammalian cells inside a support matrix cost effective with respect to the consumption of cultivation medium. In addition, the small volume of medium allows to monitor the metabolic activity of the cells and expression of tissue specific marker substances which are secreted in small amounts because these are not diluted in a large volume of medium, therefore allowing the accumulation of analytically detectable concentrations.

As a further aspect, the inventive bioreactor system can conveniently be assembled and sterilized, including the support matrix, provided its constituent materials are resistant to the irradiation, chemicals or high temperatures used for sterilization. This is because the entire bioreactor system can be provided with a peristaltic pump attaching to the circulating duct connecting the outlet port of the carrier pipe and the inlet port of the return pipe, provided medium can be filled in via a separate port or e.g. the inlet port, and sterile air for cultivation can be supplied to the medium.

Suitable materials for the matrix carrier are for example plastic resin, preferably PEEK, stainless steel and glass.

### BRIEF DESCRIPTION OF THE DRAWINGS

FIG. 1 shows a schematic cross-sectional view of a bioreactor according to the invention.

FIGS. 2a and 2b show detailed cross-sectional views of two embodiments of the matrix carrier.

FIG. 3 shows a schematic arrangement of the inventive bioreactor wherein medium drawn from the bioreactor is partly replaced by fresh medium and/or a different medium composition.

FIG. 4 shows a graph of the L-lactate production rate, lactate dehydrogenase release, and alkaline phosphatase production rate in cultivation runs performed in inventive bioreactors according to FIG. 3 with continuous partial replacement of medium.

FIG. 5 shows the concentration of osteocalcin in the supernatant as measured by ELISA and the total cell number within the support matrix as measured by DNA fluorescence staining with Hoechst 33258.

In the figures, like reference numerals designate like parts and portions.

### DETAILED DESCRIPTION OF THE INVENTION

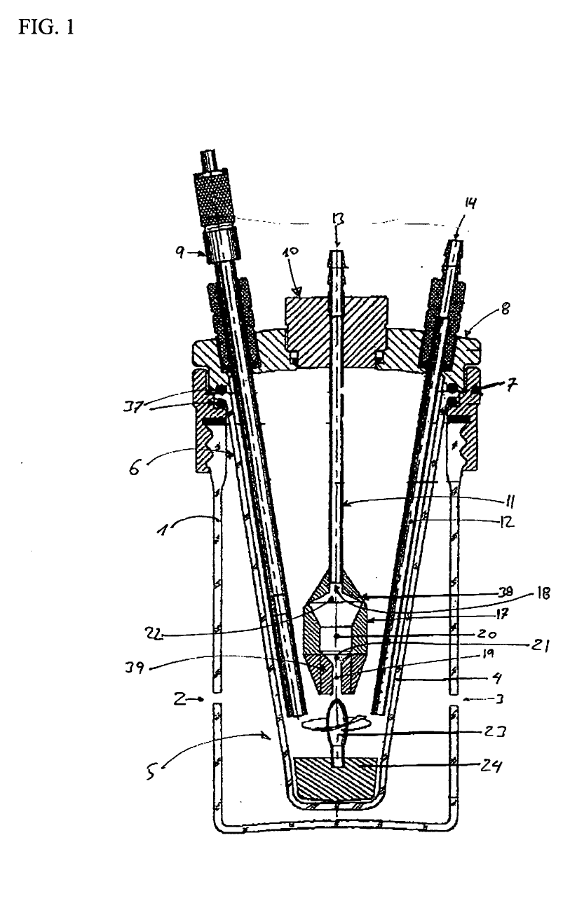

As is shown in FIG. 1, the bioreactor according to the invention comprises an outer vessel 1 having a top opening and being equipped with inlet port 2 and outlet port 3 for thermostatic fluid. Arranged within outer vessel 1 is inner vessel 4. The inner vessel 4 is formed such that the inner diameter of its portion forming the closed bottom end 5 is smaller than its diameter of the opposite top portion 6, which is open and sealable by lid 8. Preferably, the inner vessel 4 has the shape of a frustrum of a cone. The rim of the top opening of the outer vessel is covered by holding ring 7 and lid 8. Between holding ring 7 and the rim of the outer vessel 1 there is arranged a sealing. The holding ring 7 has an inner thread to fit over the outer thread of the rim. A second inner thread of the holding ring 7 is engagable with an outer thread of lid 8. The edge of the inner vessel 4 is shown to be sealingly arranged between the lid 8 and the holding ring 7, the threaded engangement of holding ring 7 and lid 8 also sealing lid 8 to inner vessel 4. Lid 8 has borings to sealingly hold penetrating probes like the measuring or sampling probe 9 and the plug element 10, sealingly holding carrier pipe 11, as well as the return pipe 12.

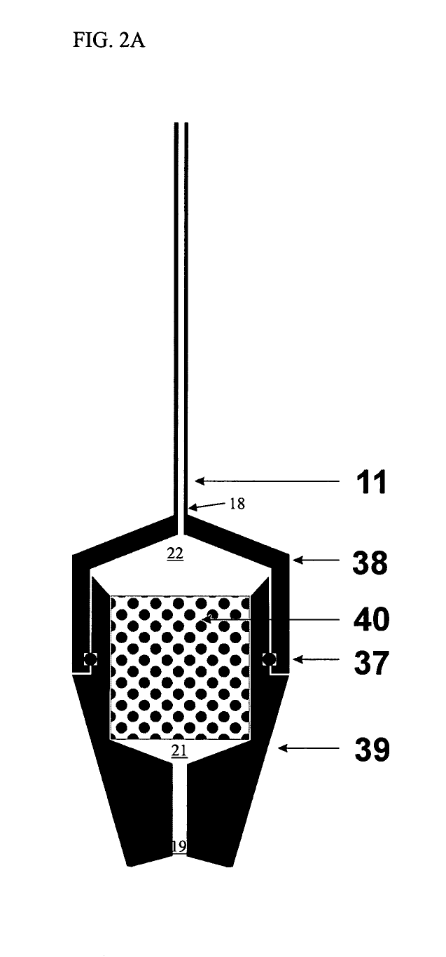

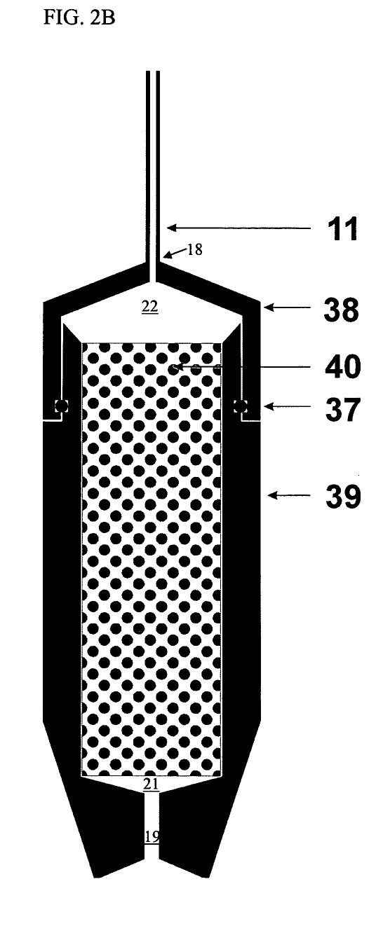

Arranged on the first end of the carrier pipe 11 opposite its second end is the matrix carrier 17, connected fluid-tight at its outlet boring 18, positioned on its first end. As can be seen in greater detail in FIG. 2, opposite the outlet boring 18 on its second end, the matrix carrier 17 has an inlet boring 19, which is open to the interior of inner vessel 4. Matrix carrier 17 encloses interior volume 20, which is accessible by outlet boring 18 and inlet boring 19. The interior volume 20 preferably has a conical inlet region 21, enlarging the inner diameter of the inlet boring 19 up to the diameter of the interior volume 20 of the matrix carrier 17. This conical inlet region 21 serves to distribute inflowing medium across the surface of the support matrix arranged within the interior volume 20 of the matrix carrier 17. The conical inlet region 21 is matched by conical outlet region 22, acting as a funnel from the interior volume 20 to the diameter of carrier pipe 11.

Preferably, the interior volume 20 of matrix carrier 17 is formed to accommodate the shape of the support matrix except for those surfaces adjacent outlet boring 18 and inlet boring 19 of the matrix carrier which may be formed by the conical inlet region 21 and conical outlet region 22.

A second end of the carrier pipe 11 is formed by an exit port 13 and return pipe 12 has an inlet port 14, with both ports connected to each other by a circulating duct 15, which may be executed completely or partially in the form of a pipeline or tubing. On or within circulating duct there is arranged circulation pump 16, drawing medium from exit port 13 of carrier pipe 11 and delivering it to inlet port 14 of return pipe 12. Circulation pump 16 may be executed as a peristaltic pump or another type of pump allowing the sterile transport of medium through circulating duct 15.

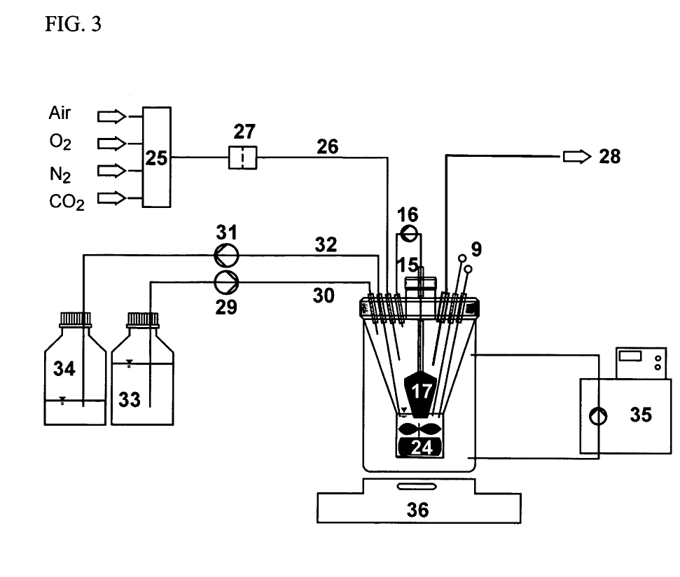

As an alternative to or in addition to circulating the medium through the inner volume of matrix carrier 17 and returning it via circulating duct 15 and inlet port 14 of return pipe 11, fresh replacement medium or medium of a different composition can fully or partially replace the medium circulating through the matrix carrier 17. An arrangement for partially replacing the cultivation medium by a different medium composition is shown in FIG. 3. There, the exchange medium is delivered from medium supply vessel 33 via feed pump 29 and feed line 30 through an additional inlet port in lid 8. Medium is partially removed by means of drain pump 31 via drain line 32 into harvest vessel 34. Thermostat 35 controls the cultivation temperature of outer vessel 1 and subsequently that of inner vessel 4 as well. As a consequence, cultivation medium is continuously partly replaced with exchange medium by the cooperation of feed pump 29, delivering exchange medium via feed line 30 and drain pump 31 acting on drain line 32, removing medium from inner vessel 4.

Practical use of the bioreactor have shown that a partial replacement of 12 up to 5 times the volume of medium per 24 hours cultivation is sufficient for supplying the cells with the necessary nutrients.

An arrangement for using the present invention without circulating at least part of the cultivation medium uses comparatively large quantities thereof. Preferably, at least part of the cultivation medium exiting inner vessel 4 is returned to the interior volume 20 of the matrix carrier 17 via return pipe 12. Most preferably, the cultivation medium is circulated without partially or fully replacing it until at least one of its ingredients reaches a limiting level or until a different medium composition is desired. As an example, different cell growth factors like cytokines can to be replaced for influencing the growth or differentiation behavior by means of a continuous partial replacement of medium by the exchange medium, introducing the exchange medium via feed line 30 and removing medium via drain line 32.

FIGS. 2a and 2b schematically depict embodiments of the matrix carrier in detail. The matrix carrier is attached to carrier pipe 11, for example by engaging threads. The matrix carrier 17 is shown as being constituted of two parts, namely an upper part 38 which is attached to the carrier pipe 11 via its outlet boring 18. Upper part 38 is shown in a state fastened to lower part 39, open towards inner vessel 4 via its inlet boring 19. For better sealing off the interior volume 20 of matrix carrier 17 enclosed by upper part 38 and lower part 39 a sealing 37 is positioned between the two parts 38 and 39. The sealing 37 may be an elastic sealing like an O-ring. The predominant portion of the interior volume 20 of matrix carrier 17 is filled with support matrix 40, which is held therein with positive fit on its surfaces perpendicular to the faces dircetly opposite the inlet 19 and outlet 18 borings.

The design of the matrix carrier can be freely chosen as long as the perfusion of the support matrix with medium entering through inlet boring 19 and leaving via outlet boring 18 is ensured by the positive fit at least along a full circumferential line between the two borings 18 and 19. Preferably, the matrix carrier 17 is designed to avoid interior volume 20 not to be filled by support matrix 40.

In FIG. 3, the stirrer drive 36 is used to move the magnetic stirrer carrier 24 of stirrer 23. Gases are introduced via gas mixing unit 25 and sterile filter 27.

As a further embodiment of using the inventive bioreactor, the cultivation medium exiting the carrier pipe 11 is conditioned before being returned via return pipe 12. For example, such conditioning can be achieved by a sparger or via an additional inlet port for medium components integrated into the circulating duct 15.

In operation, the cell cultivation medium inside the inner vessel 4 should attain a minimum level such that the inlet boring 19 of the matrix carrier 17 is submersed in order to allow medium to be drawn into the interior volume 20 of the matrix carrier 4.

As an optional component of the inventive bioreactor, a stirrer 23 is arranged on the rotation axis of magnetic stirrer carrier 24. Apart from conveying the kinetic energy of a magnetic stirrer 36 arranged below the bioreactor, the stirrer carrier 24 preferably serves to displace part of the interior volume of the inner vessel 4 at its bottom region in order to minimize the volume of medium necessary. In the alternative, although less preferred, the inventive bioreactor can use the flow of medium exiting the return pipe 12 to provide for mixing of cultivation medium before entering the inlet boring 19 of matrix carrier 17.

### EXAMPLE

The inventive bioreactor system can be used for the generation of bone tissue implants in vitro. For that purpose cells with the potential of generating an extracellular matrix with subsequent mineralisation were cultivated three-dimensionally on a suitable scaffold material serving as the support matrix, which is biocompatible and bioresorbable. Osteogenic cells isolated from femura of 5 week old female rats were allowed to settle on macroporous beta-tricalcium-phosphate scaffold structures having interconnecting pores. A total of 10 million cells were seeded onto each scaffold material having a total volume of 1 cm³ and allowed to attach to the material for 12 hours before being introduced into the matrix carrier 17 of the bioreactor.

For long-term cultivation of osteogenic cells for more than 7 days a continuous partial medium exchange was found to be preferable compared to a batchwise medium exchange. Despite of a more complex set-up of the medium supply to the inventive bioreactor this cultivation mode reduces the required amount of handling steps during the cultivation and thus minimises the risk of contamination. The additional set-up comprises a medium supply vessel 33 and a corresponding feed pump 29 to pump exchange medium via feed line 30 into the inner vessel 4 as well as a drain pump 31, drawing medium out of inner vessel 4 by means of drain line 32 into harvest vessel 34. In addition, this mode provides more steady state culture conditions which better reflect the situation in vivo.

Two different cultivation media were used during each cultivation process. During the first 7 days, a proliferation medium was used for continuous replacement of the medium drawn from drain line 32 by exchange medium introduced via feed line 30. From day 7 to the end of the cultivation period, a differentiation medium was introduced using the same procedure for gradual medium exchange. With this replacement medium the cytokines beneficial for cell proliferation which on the other hand reduce the differentiation potential are gradually removed in order to reduce cell growth and rather support cell differentiation. A total of 10 ml of medium was exchanged per day, corresponding approximately to the interior volume of inner vessel 4.

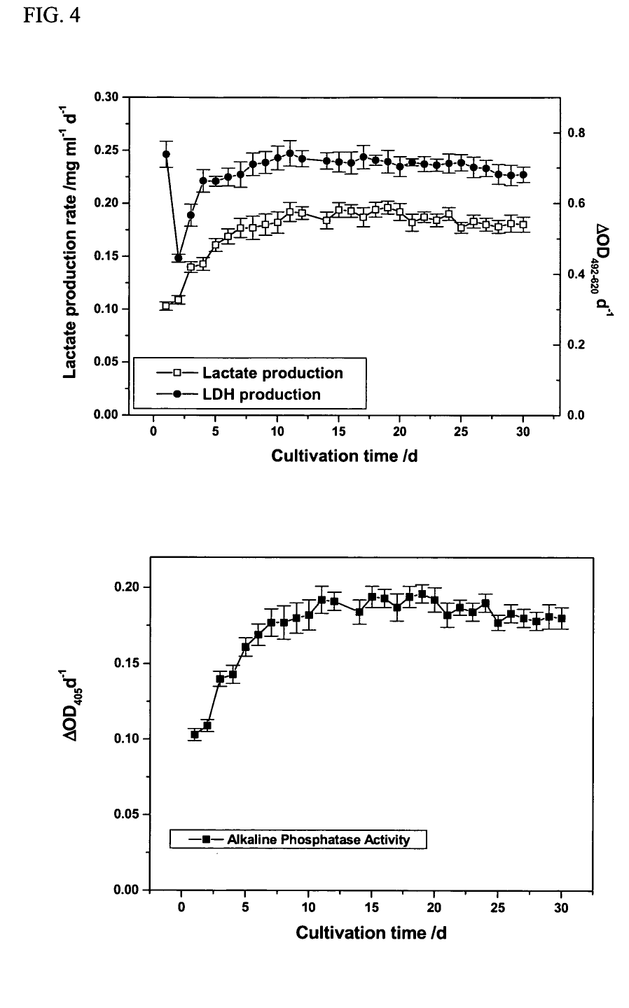

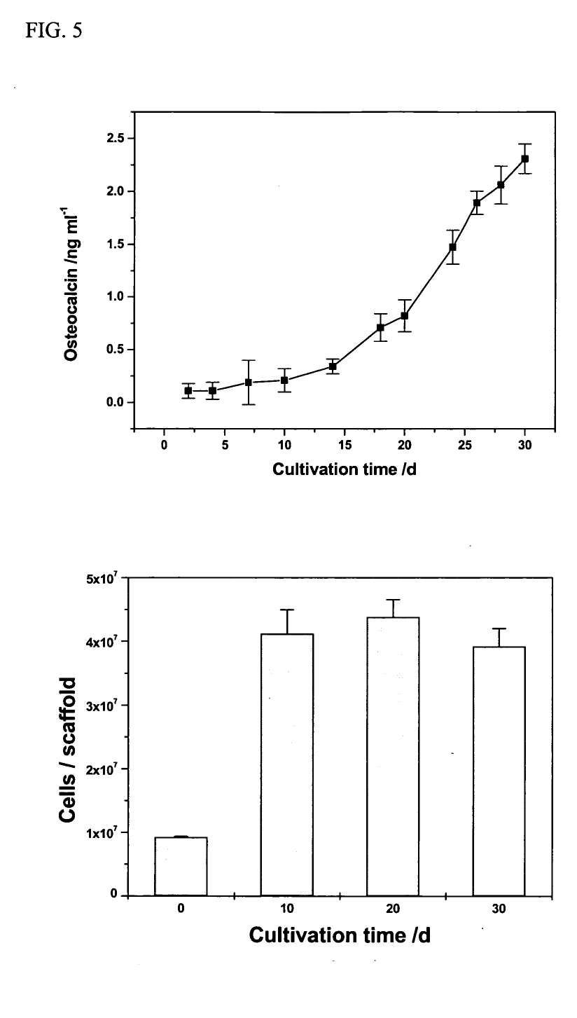

For the investigation of cell proliferation and differentiation under these culture conditions a number of parameters was monitored. The results are shown in FIGS. 4 and 5. The production of L-lactate reflects the overall metabolic activity of cells, soluble alkaline phosphatase and osteocalcin represent predominant markers for osteogenic differentiation, and the release of lactate dehydrogenase reflects cell death. The total number of cells was measured by DNA fluorescence staining using the stain Hoechst 33258. This analysis is an invasive method requiring the homogenisation of support matrix and cells.

The analytical results shown in FIG. 5, especially the L-lactate production rate, demonstrate that there is an initial rapid cell growth followed by a stationary phase, indicating that cells have completely covered the macroporous surface of the scaffold and then have entered the phase of differentiation. This is confirmed by the production rate of soluble alkaline phosphatase, also shown in FIG. 4, and finally by the production of osteocalcin which is associated with mineralisation (see FIG. 5). The total cell number initially increases by a factor of 4, then remains constant during the remaining course of the cultivation (see FIG. 5). These results can be interpreted as reflecting the state of confluency that the cells have reached on the support matrix and then differentiated into mature, non-proliferating cells along with the synthesis of an extracellular matrix.

As indicated by the constant release of lactate dehydrogenase (LDH) there is a steady lysis of a small amount of cells which are apparently replaced by new ones. A similar behaviour is also observed in conventional two-dimensional culture. The high concentration of LDH at the beginning is caused by cells which are non-attached during the first 12 hours before the support matrix is transferred into the matrix carrier 17 bioreactor. With the onset of perfusion of the support matrix with cultivation medium these cells are flushed out of the support matrix and lyse, as is reflected by the release of LDH (see FIG. 4).

In summary, the cultivation process is demonstrated to be highly suitable for the three-dimensional propagation of osteogenic cells in vitro and their subsequent differentiation on biocompatible and bioresorbable support materials.

List of Reference Numerals:

Outer vessel 1

Inlet port 2

Outlet port 3

Inner vessel 4

Bottom portion of inner vessel 5

Top portion of inner vessel 6

Holding ring 7

Lid 8

Probe 9

Plug element 10

Carrier pipe 11

Return pipe 12

Exit port 13

Inlet port 14

Circulating duct 15

Circulation pump 16

Matrix carrier 17

Outlet boring 18

Inlet boring 19

Interior volume 20 of matrix carrier

Conical inlet region 21

Conical outlet region 22

Stirrer 23

Magnetic stirrer carrier 24

Gas mixing unit 25

Gas supply line 26

Sterile filter 27

Gas exhaust line 28

Feed pump 29

Feed line 30

Drain pump 31

Drain line 32

Medium supply vessel 33

Harvest vessel 34

Thermostat 35

Stirrer drive 36

Sealing 37

Upper part 38 of matrix carrier 17

Lower part 39 of matrix carrier 17

Support matrix 40

1. A bioreactor for cultivating mammalian cells within a porous support matrix within a cultivation medium, comprising an inner vessel (4) disposed within an outer vessel (1), said inner vessel (4) having a smaller inner diameter at its closed bottom portion and a larger inner diameter at its opposite open top portion, and a lid (8) sealingly covering the top portion of said inner vessel (4), said lid (8) sealingly accommodating penetrating probes, pipes, and supply ducts,
wherein said bioreactor further comprises a matrix carrier (17) arranged in fluid proof connection at a first end of a carrier pipe (11), the opposite second end of said carrier pipe (11) sealingly penetrating said lid (8), the matrix carrier (17) having an inlet boring (19) at its first end open to the volume of said inner vessel (4) and an outlet boring (18) at its second end opposite its first end, the second end connected in fluid proof connection to the first end of said carrier pipe (11),
the interior volume (20) of said matrix carrier (17) essentially shaped to accommodate faces of a support matrix at least on a circumferential line thereof by positive fit for ingrowth of mammalian cells while allowing medium to enter said interior volume (20) via inlet boring (19) and leave via outlet boring (18), and
a return pipe (12) being in fluid proof connection with said carrier pipe (11) for circulating at least part of the cultivation medium.

2. The bioreactor according to claim 1, wherein said return pipe (12) is connected to said carrier pipe (11) via a circulating duct (15), to which a circulation pump (16) is attached for pumping medium through matrix carrier (17) and carrier pipe (11) back into inner vessel (4) via return pipe (12).

3. The bioreactor according to claim 1, further comprising a feed line (30) for delivering medium from a medium supply vessel (33) into inner vessel (4) and a drain line (32) for removing medium from inner vessel (4).

4. The bioreactor according to claim 1, further comprising a feed line (30) connected to the circulation duct (15) for feeding medium therein and a drain line (32) branching off the circulation duct (15) for removing medium therefrom.

5. The bioreactor according to claim, 3, further comprising a level control probe to monitor the level of medium inside inner vessel (4) and regulate the flow of medium in said feed line (30) and said drain line (32).

6. (cancelled)

7. A method for preparing osteoblastic cells, said method comprising cultivating osteoblastic cells within the body of a porous support matrix of a bioreactor according to claim 1.

8. The bioreactor according to claim 4, further comprising a level control probe to monitor the level of medium inside inner vessel (4) and regulate the flow of medium in said feed line (30) and said drain line (32).
